(19) United States
(12) Patent Application Publication
(10) Pub. No.: US 2020/0108006 A1
(43) Pub. Date: Apr. 9, 2020

(54) Human-Derived Keratin Hair Filler Compositions and Methods of Use

(71) Applicant: Virtue Labs, LLC, Winston-Salem, NC (US)

(72) Inventors: William JACOBSEN, Grafton, MA (US); Erin FALCO, Winston-Salem, NC (US); Traci FARMER, Raleigh, NC (US); Tiffany HENDLEY, Greensboro, NC (US)

(21) Appl. No.: 16/592,998

(22) Filed: Oct. 4, 2019

Related U.S. Application Data
(60) Provisional application No. 62/741,150, filed on Oct. 4, 2018.

(51) Int. Cl. A61K 8/65; A61Q 5/06; A61Q 5/00; A61K 8/02; A61Q 5/02; A61Q 5/12; A45D 19/00

(52) U.S. Cl. CPC A61K 8/65; A61Q 5/065; A61Q 5/002; A61K 8/022; A45D 2019/0066; A61Q 5/12; A45D 19/00; A61K 2800/84; A45D 2019/0058; A61Q 5/02

(57) ABSTRACT

Keratin protein filler compositions are provided that include at least one keratin protein derived from human hair. Hair coloring kits are also provided that include at least one keratin protein filler composition and at least one hair colorant composition. Methods of treatment of various hair conditions are also provided.

### RELATED APPLICATION

This application claims the benefit of U.S. Provisional Patent Application Ser. No. 62/741,150 filed Oct. 4, 2018, which is incorporated by reference in its entirety.

### BACKGROUND

Keratins are fundamental compounds of the skin, the hair, the eyelashes and the nails. These fibrous proteins contribute towards their form, elasticity and strength. Hair is a unique source of human keratins because it is one of the few human sources that are readily available, inexpensive and provides protein structure equivalent to hair.

Environmental factors such as sunlight, blow drying, flat ironing, combing, brushing, exposure to harsh condition (chlorine in pools) colorants and chemicals related to styling and the coloring process deteriorate and weaken the hair as well as destroy the structure and the surface of hair. All of these factors stress and damage the hair. Particularly, the coloring process intentionally stresses and swells the hair to allow the dye molecules to penetrate the shaft and impart or remove color. As hair is damaged the surface of hair is compromised enabling the loss of protein and moisture, so the hair is left brittle and more prone to breakage. As a result, the hair dries and becomes brittle, split ends are formed or the hair may break. Hair constituent proteins may be removed by treatments with cosmetics such as shampoos, perm chemicals, or hair dyes. As a result, the hair is thinner and the likelihood of hair damage increases. Hair is unable to restore itself to its original state without assistance.

Accordingly, there remains a need for a hair product that repairs hair that was previously colored or combined with a hair colorant composition to repair as well as prevent damage to hair.

### SUMMARY

According to one aspect, a keratin protein filler composition for colored hair is provided. The keratin protein filler composition includes at least one keratin protein derived from human hair. The keratin protein may be formulated as a powder, gel, paste or cream. According to one embodiment, the keratin protein includes one or more of alpha-keratin, gamma-keratin, or a combination thereof. According to one embodiment, the keratin protein includes at least one kerateine such as, for example, alpha-kerateine, gamma-kerateine, or a combination thereof. According to one embodiment, the powder is formed by a process that includes the steps of flash freezing the keratin protein derived from human hair to form a frozen cake, drying the frozen cake, and pulverizing the cake to form the keratin protein powder. According to one embodiment, the keratin protein is not denatured and maintains natural keratin protein conformation.

According to one aspect, a hair colorant kit is provided. The hair colorant kit includes at least one keratin protein filler composition, at least one hair colorant composition, and at least one set of instructions for use. According to one embodiment, the kit further includes a colorant developer component. According to one embodiment, the colorant composition includes one or more compounds to impart temporary or semi-permanent color hair. According to one embodiment, the colorant composition optionally includes a bleaching compound to make the hair a lighter shade upon introduction. According to one embodiment, the colorant composition optionally includes one or more dyes or pigments to impart a desired hair color. According to one embodiment, the colorant composition optionally includes one or more oxidizing or complexing agents, dyes or pigments, coupling compounds (i.e., couplers), ammonia, fatty alcohols, emulsifying agents, surfactants color precursors alkalizing agents, antioxidants, or perfumes. According to one embodiment, the colorant kit may optionally include at least one of a shampoo, a hair conditioner, or a combination thereof. The shampoo and conditioner may optionally include pink pomelo, hydrolyzed quinoa, artichoke leaf, vitamin A, vitamin C, vitamin B1, zinc, gotu kola, tapioca starch, kaolin clay, pea protein, phospholipids, brown algae, silica silylate, hydrated silica, asiaticoside/madecassoside, panthenol, cypress, baobab seed oil, or any combination thereof.

According to one aspect, a method of repairing and coloring hair is provided. According to one embodiment, the hair is repaired by filling in damaged areas on a hair strand with the hair filler composition as provided herein. The repair and coloring method includes the steps of mixing a hair colorant composition with a keratin protein filler composition to form a hair colorant mixture and applying the hair colorant mixture to a user's hair to both color and repair the hair in a contemporaneous manner (i.e., at the approximate same time). The hair colorant mixture may be in the form of a paste, gel, or cream. According to one embodiment, the method further includes the step of mixing a developer component with the hair colorant composition prior to mixing with the keratin protein filler composition. According to one embodiment, the hair colorant mixture refills any keratin that may be eroded from the hair thereby strengthening and thickening the hair. The hair colorant mixture repairs any existing damage to the hair and prevents damage to the hair that might be caused by application of the hair colorant composition. Thus, the resulting colored hair is healthier than that prior to application and not damaged as is typical upon application of harsh colorant compounds to hair.

According to one aspect, a method for reducing breakage of colored hair is provided. The method includes the steps of applying a hair colorant mixture as provided herein to a surface of the hair in need of treatment and rinsing the hair to remove the hair colorant mixture thereby increasing hair thickness to reduce hair breakage of the colored hair.

According to one aspect, a method of reducing hair frizziness in colored hair is provided. The method includes the steps of applying a hair colorant mixture as provided herein to a surface of the hair in need of treatment and rinsing the hair to remove any excess hair colorant mixture thereby causing the hair to exhibit less frizz in the colored hair.

According to another aspect, a method of increasing hair strand thickness of colored hair is provided. The method includes the step of administering, applying or otherwise introducing the keratin protein filler composition to the surface of a mammal's hair in need of treatment. The hair may be previously colored or colored at the time of administration.

According to one aspect, a method for reducing breakage of or damage to hair is provided. The hair may be colored or natural, non-colored hair. The method includes the steps of applying to a surface of the hair in need of treatment a hair treatment composition, the hair treatment composition including one or more of a shampoo, conditioner, mask, or paste and a keratin protein filler composition. Upon application, hair thickness is increased to reduce hair breakage and damage to the hair. According to one embodiment, the method further includes the step of rinsing the hair to remove the hair treatment composition. According to one embodiment, the hair treatment composition is rinsed off immediately upon application. According to one embodiment, the hair treatment composition is left in contact with the hair for up to ten minutes, twenty minutes, or thirty minutes prior to rinsing. According to another embodiment, the hair treatment composition is left in contact with the hair indefinitely.

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, the terms “treated” and “colored” may be used interchangeably and refer to hair that has had applied thereto one or more chemical components, dyes, pigments, or other hair products commonly applied to hair that may result in damage to the hair.

As used herein, the term “damaged” may refer to hair that has one or more imperfections including, but not limited to, brittleness, frizziness, weakened or thinning areas on the hair strand (i.e., craters in the hair strand or cuticle), lack of vibrancy, lack of sheen or shine, lack of volume, or any combination thereof.

As used herein, the term “keratin protein source” includes proteinaceous sources of keratin proteins from human hair. The human hair can be end-cut, as one would typically find in a barber shop or salon.

As used herein, the term “keratin protein(s)” as used herein collectively refers to hair protein sources, including but not limited to naturally occurring keratin, reduced keratin, oxidized keratin, S-sulfonated keratin, or a combination thereof. This term also refers to the extracted keratin derivatives that are produced by oxidative and/or reductive treatment of keratin, including but not limited to keratose, alpha-keratose, gamma-keratose, kerateines, alpha-kerateine, or gamma-kerateine. Soluble keratins can be extracted from human hair fibers by oxidation or reduction using methods known in the art. If one employs an oxidative treatment, the resulting keratins are referred to as “keratoses.” If a reductive treatment is used, the resulting keratins are referred to as “kerateines.”

The term “alpha keratin” as used herein refers to a type of keratin protein that exhibits a molecular weight range from about 60 Da to about 6.2 MDa.

According to one embodiment, a hair filler composition is provided. The hair filler composition may repair existing hair damage incurred from hair colorants and any related chemicals that may damage the hair during the coloring process. The hair filler composition may also prevent further damage associated with the hair coloring processing. According to a particular embodiment, the hair filler composition repaves the cuticle of the hair and refills any keratin that was eroded by previous or current hair coloring processes. The hair filler composition prevents hair breakage, increases vibrancy of the hair color, aids in any existing color coverage (e.g., gray color coverage), and, when combined with a hair colorant composition as provided herein to form a colorant paste and applied to hair, actually results in colored hair that is healthier than the hair was prior to the coloring process. While not being bound to a particular theory, the hair filler composition becomes reactive as soon as the hair filler composition meets or comes in contact with one or more dyes, pigments or bleaching components. The hair filler composition is drawn directly to any point in a hair strand that is weakened and refills or otherwise repairs the hair. The hair strand is fortified or otherwise strengthened. Any dye or pigment present in an accompanying hair colorant composition as provided herein adheres to the hair more completely and binds more tightly, for richer, longer lasting color. The hair filler composition as provided herein also provides, healthier, softer, and shinier hair upon use.

According to one embodiment, the hair filler composition as provided herein includes keratin protein. According to a particular embodiment, the hair filler composition as provided herein includes keratin protein in a reduced keratin form (kerateine protein). According to one embodiment, the hair filler composition is devoid of any sulfates, parabens, phthalates, colors, dyes, or gluten.

The keratin protein as provided here may be extracted according to the methods as provided herein. The method includes the step obtaining or providing a keratin protein source. The keratin protein source is human hair. According to one embodiment, the human hair is of Asian origin. The keratin protein source is not derived from other sources such as, for example, wool, fur, horns, hooves, beaks, feathers, scales. According to one embodiment, at least one reductant agent is added to the hair and mixed. The reduced hair is then rinsed to remove residual reductant agent. According to one embodiment, the rinsed hair is then washed in an aqueous solution to release the water soluble proteins from the hair into a liquid extract.

According to one embodiment, the extraction method further includes the step of separating and purifying the liquid extract resulting in a soluble keratin protein solution. According to one embodiment, the keratin protein includes one or more of alpha-keratin, gamma-keratin, or a combination thereof. According to one embodiment, the keratin protein includes at least one kerateine such as, for example, alpha-kerateine, gamma-kerateine, or a combination thereof.

According to a particular embodiment, the keratin protein derived according to the extraction methods as provided herein is then frozen to form a frozen keratin protein cake. According to one embodiment, the keratin protein is flash frozen to form a flash frozen keratin protein cake. According to one embodiment, the step of flash freezing the keratin protein is carried out under controlled freeze rate conditions. According to one embodiment, the keratin protein is flash frozen in a controlled rate manner over a period of time of about 3 hours or less. According to one embodiment, the keratin protein is flash frozen in a controlled rate manner over a period of time of about three hours or less. According to one embodiment, the keratin protein is flash frozen in a controlled rate manner over a period of time of about three hours.

According to one embodiment, the step of flash freezing the keratin protein is carried out at a temperature of less than about −20° C. According to another embodiment, the step of flash freezing the keratin protein is carried out at a temperature of less than about −40° C. According to another embodiment, the step of flash freezing the keratin protein is carried out at a temperature of less than about −60° C. According to another embodiment, the step of flash freezing the keratin protein is carried out at a temperature of less than about −80° C. According to another embodiment, the step of flash freezing the keratin protein is carried out at a temperature of less than about −90° C. According to a particular embodiment, the step of flash freezing the keratin protein is carried out at a temperature of less than about −80° C. According to a particular embodiment, the step of flash freezing the keratin protein is carried out at a temperature of less than about −120° C. According to a particular embodiment, the step of flash freezing the keratin protein is carried out at a temperature of less than about −160° C. According to a particular embodiment, the step of flash freezing the keratin protein is carried out at a temperature of less than about −196° C. According to a particular embodiment, the step of flash freezing the keratin protein is carried out at a temperature of about −60° C. and about −196° C. According to a particular embodiment, the step of flash freezing the keratin protein is carried out at a temperature of about −60° C. and about −86° C.

According to one embodiment, the step of flash freezing is carried out by introducing liquid nitrogen to the keratin protein.

According to one embodiment, the flash frozen keratin protein cake is then dried. After drying, the dried flash frozen protein cake is, ground, minced, or otherwise pulverized for form a plurality of particles (i.e., a powder). The powder particles are of a size of from about 20 to about 2000 um.

According to one embodiment, the keratin protein as provided herein is not precipitated during production of the keratin protein composition. According to one embodiment, the keratin protein maintains a naturally occurring shape or conformation (i.e., does not undergo any transformation or denaturing) during extraction from human hair and subsequent processing. According to one embodiment, the keratin protein does not undergo any transformation or denaturing during extraction from human hair and subsequent processing. The extraction process ensures the harvested proteins are of correct conformational structure. According to one embodiment, the keratin proteins as provided herein are substantially or completely soluble in water.

According to one embodiment, a hair colorant kit is provided. According to one embodiment, the hair colorant kit includes a hair filler composition as provided herein. According to one embodiment, the colorant kit further includes a colorant composition. The colorant composition includes one or more compounds for imparting temporary or semi-permanent color hair. According to one embodiment, the colorant composition includes a bleaching compound to make the hair lighter or blonde upon introduction. The colorant composition may include at least one oxidizing or complexing agents such as, for example, hydrogen peroxide. The colorant composition may include one or more dyes or pigments to impart a desired hair color. The one or more dyes or pigments may be utilized to cover gray or other undesired colored hairs. The colorant composition may include one or more coupling compounds (i.e., couplers) to impart a particular shade of the desired color. The colorant composition may further include ammonia. The colorant composition may further include a base cream that is an emulsion of an oil and water component, one or more fatty alcohols, emulsifying agents, and surfactants. The colorant composition may further include one or more color precursors such as, for example, para-toluylenediamenesulphate, resorcinol, or meta-aminophenol. The colorant composition may further include one or more alkalizing agents, antioxidants, or perfumes. The color composition may also include one or more ingredients that do not impact or change the shape of conformation of the keratin protein.

According to one embodiment, the hair colorant kit may optionally include at least one of shampoo, conditioner, mask, or a combination thereof. The shampoo, conditioner or mask may optionally include pink pomelo, hydrolyzed quinoa, artichoke leaf, vitamin A, vitamin C, vitamin B1, zinc, gotu kola, tapioca starch, kaolin clay, pea protein, phospholipids, brown algae, silica silylate, hydrated silica, asiaticoside/madecassoside, panthenol, cypress, baobab seed oil, or any combination thereof. The shampoo, conditioner, or mask may also optionally include tocopherol, dimethicone, parabens, titanium dioxide, sodium lauryl sulphate, sodium laureth sulphate, retinol, collagen, ambergris, squalene, cochineal dye, guanine, tallow, gelatin, lanolin, citric acid, sodium citrate, cocamide, guar gum, xanthum gujm, glycol distearate, polyglycol esters, sodium chloride, glycerin, cetyl alcohol, stearyl alcohol, panthenol, silicones, or any combination thereof. The colorant composition may also include one or more of D-panthenol, polysorbate 20, cetyl alcohol, Crambe abyssinica seed oil, hydroxyethyl acrylate/sodium acryloyldimethyl taurate copolymer, isohexadecane, polysorbate 60, cyclopentasiloxane, cyclohexasiloxane, 011-13 isoparaffin, dimethiconol, isohexadecane, dimethicone, caprylyl glycol, phenoxyethanol, hexylene glycol, or any combination thereof. The colorant composition may also include on or more of the cosmetic additives, polymers, solvents, or film forming agents as described herein.

According to one embodiment, the hair colorant kit includes at least one developer component or hair crème component. The at least one developer component helps the hair colorant composition to oxygenate and mix together to create the desired color shade. Particularly, the developer aids in the color attachment to the hair shaft. The developer component may be formulated as a gel, paste or cream.

According to one embodiment, the hair colorant kit includes at least one set of instructions.

According to another aspect, a method for reducing hair breakage of colored hair is provided. The method includes the step of administering, applying or otherwise introducing the keratin protein filler composition as provided herein to a surface of the hair in need of treatment thereby increasing hair thickness and hair volume to reduce breakage of colored hair. The hair may be previously colored or colored at the time of administration. The hair may be styled immediately after the coloring process is completed.

According to another aspect, a method of reducing hair frizziness of colored hair is provided. The method includes the step of administering, applying or otherwise introducing the keratin protein filler composition as provided herein to a surface of the hair in need of treatment thereby causing the hair to exhibit less frizz in colored hair. The hair may be previously colored or colored at the time of administration.

According to another aspect, a method of increasing hair strand thickness of colored hair is provided. The method includes the step of administering, applying or otherwise introducing the keratin protein filler composition to the surface of a mammal's hair in need of treatment. The hair may be previously colored or colored at the time of administration.

According to another aspect, a method of increasing hair strand volume of colored hair is provided. The method includes the step of administering, applying or otherwise introducing the keratin protein filler composition to the surface of a mammal's hair in need of treatment. The hair may be previously colored or colored at the time of administration.

According to another aspect, a method of retaining hair gloss in colored hair is provided. The method includes the step of administering, applying or otherwise introducing the keratin protein filler composition to the surface of a mammal's hair in need of treatment. The hair may be previously colored or colored at the time of administration.

According to another aspect, a method of improving hair color concentration is provided. The method includes the step of administering, applying or otherwise introducing the keratin protein filler composition to the surface of a mammal's hair in need of treatment. The hair may be previously colored or colored at the time of administration.

According to one embodiment, a method of reducing skin irritation associated with the hair coloring process is provided. The skin irritation is typically caused by scalp exposure to one or more of the chemical components typically found in the skin colorant composition. The method includes the step of administering, applying or otherwise introducing the hair colorant mixture as provided herein to the surface of a mammal's hair that is in need of treatment. The keratin protein filler composition aids in reducing or otherwise eliminating any sort of irritation (itching, burning, etc.) that is associated with the hair coloring process.

According to another aspect, a method for reducing hair breakage of or damage to colored hair is provided. The method includes the step of administering, applying or otherwise introducing the keratin protein filler composition as provided herein to a surface of the hair in need of treatment thereby increasing hair thickness and hair volume to reduce breakage of colored hair. The hair may be previously colored or colored at the time of administration. The hair may be styled immediately after the coloring process is completed.

According to another aspect, a method of reducing hair frizziness of natural or non-colored hair is provided. The method includes the step of administering, applying or otherwise introducing the keratin protein filler composition as provided herein to a surface of the hair in need of treatment thereby causing the hair to exhibit less frizz.

According to another aspect, a method of increasing hair strand thickness of natural or non-colored hair is provided. The method includes the step of administering, applying or otherwise introducing the keratin protein filler composition to the surface of a mammal's hair in need of treatment.

According to another aspect, a method of increasing hair strand volume of natural or non-colored hair is provided. The method includes the step of administering, applying or otherwise introducing the keratin protein filler composition to the surface of a mammal's hair in need of treatment.

According to another aspect, a method of retaining hair gloss in natural or non-colored hair is provided. The method includes the step of administering, applying or otherwise introducing the keratin protein filler composition to the surface of a mammal's hair in need of treatment.

According to one aspect, a method for reducing breakage of or damage to hair is provided. The hair may be colored or natural, non-colored hair. The method includes the steps of applying to a surface of the hair in need of treatment a hair treatment composition, the hair treatment composition including one or more of a shampoo, conditioner, mask, or paste and a keratin protein filler composition as provided herein. Upon application, hair thickness is increased to reduce hair breakage and damage to the hair. According to one embodiment, the method further includes the step of rinsing the hair to remove the hair treatment composition. According to one embodiment, the hair treatment composition is rinsed off immediately upon application. According to one embodiment, the hair treatment composition is left in contact with the hair for up to ten minutes, twenty minutes, or thirty minutes, or more prior to rinsing. According to another embodiment, the hair treatment composition is left in contact with the hair indefinitely (not removed by rinsing within a set time period). The method for reducing breakage of or damage to hair may further include the step of mixing the keratin protein filler composition with one or more of a shampoo, conditioner, mask, or paste to form the hair treatment composition before application to the surface of the hair. The keratin protein filler composition may be formulated as a powder.

The shampoo, conditioner or mask utilized in the method for reducing breakage of or damage to hair may be any commercially available shampoo, conditioner, or mask. The shampoo, conditioner or mask utilized in the method for reducing breakage of or damage to hair may optionally include pink pomelo, hydrolyzed quinoa, artichoke leaf, vitamin A, vitamin C, vitamin B1, zinc, gotu kola, tapioca starch, kaolin clay, pea protein, phospholipids, brown algae, silica silylate, hydrated silica, asiaticoside/madecassoside, panthenol, cypress, baobab seed oil, or any combination thereof. The shampoo, conditioner, or mask may also optionally include tocopherol, dimethicone, parabens, titanium dioxide, sodium lauryl sulphate, sodium laureth sulphate, retinol, collagen, ambergris, squalene, cochineal dye, guanine, tallow, gelatin, lanolin, citric acid, sodium citrate, cocamide, guar gum, xanthum gujm, glycol distearate, polyglycol esters, sodium chloride, glycerin, cetyl alcohol, stearyl alcohol, panthenol, silicones, or any combination thereof.

According to another aspect, a hair treatment kit is provided. The hair treatment kit includes one or more shampoo, conditioner or mask as provided herein. The hair treatment kit also includes a keratin protein filler composition as provided herein. According to such an embodiment, the keratin protein filler composition and one or more shampoo, conditioner or mask are mixed together to form a hair treatment composition for application to hair. According to one embodiment, the keratin protein filler composition is a powder. According to an alternative embodiment, the kit includes a pre-prepared hair treatment composition, the hair treatment composition including one or more of a shampoo, conditioner, mask, or paste as provided herein and a keratin protein filler composition as provided herein. According to such an embodiment, the hair treatment composition is ready for direct application to hair without any pre-mixing. The kits as provided herein optionally include a set of instructions. The kit may be utilized as provided herein on both colored and natural, non-colored hair.

All publications, patents and patent applications cited in this specification are incorporated herein by reference for the teaching to which such citation is used.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A keratin protein filler composition for colored, treated, bleached or damaged hair comprising:
at least one keratin protein derived from human hair, wherein the keratin protein is formulated as a powder, gel, paste or cream for introduction to colored hair.

2. The keratin protein filler composition of claim 1, wherein the keratin protein comprises one or more of alpha-kerateine, gamma-kerateine, or a combination thereof.

3. The keratin protein filler composition of claim 1, wherein the powder is formed by a process that includes the steps of:
flash freezing the keratin protein derived from human hair to form a frozen cake;
drying the frozen cake; and
pulverizing the frozen cake to form the keratin protein powder.

4. The keratin protein filler composition of claim 1, wherein the keratin protein is not denatured and maintains natural keratin protein conformation.

5. A hair colorant kit comprising
at least one keratin protein filler composition as provided in claim 1;
at least one hair colorant composition; and
optionally, at least one set of instructions for use.

6. The hair colorant kit of claim 5, further comprising a colorant developer component.

7. The hair colorant kit of claim 5, wherein the hair colorant composition includes one or more compounds to impart temporary or semi-permanent color hair.

8. The hair colorant kit of claim 5, wherein the hair colorant composition optionally includes a bleaching compound to make the hair a lighter shade upon introduction.

9. The hair colorant kit of claim 5, wherein the hair colorant composition optionally includes one or more dyes or pigments to impart a desired hair color.

10. The hair colorant kit of claim 5, further comprising at least one shampoo, at least one hair conditioner, or a combination thereof.

11. The hair colorant kit of claim 5, wherein the hair colorant composition optionally includes one or more oxidizing agents, complexing agents, dyes, pigments, coupling compounds, ammonia, fatty alcohols, emulsifying agents, surfactants color precursors alkalizing agents, antioxidants, perfumes, or a combination thereof.

12. A method of repairing and coloring hair, the method comprising the steps of:
mixing a hair colorant composition with a keratin protein filler composition to form a hair colorant mixture; and
applying the hair colorant mixture to a user's hair to both color and repair the hair contemporaneously.

13. The method of claim 12, further comprising the step of mixing a developer component with the hair colorant composition prior to mixing with the keratin protein filler composition.

14. The method of claim 12, wherein hair colorant mixture refills any keratin that may be eroded from the hair thereby strengthening and thickening the hair.

15. The method of claim 12, wherein the hair colorant mixture repairs any existing damage to the hair and prevents damage to the hair that might be caused by application of the hair colorant composition.

16. A method for reducing breakage of or damage to colored hair, the method comprising the steps of:
applying the hair colorant mixture of claim 12 to a surface of the hair in need of treatment; and
rinsing the hair to remove the hair colorant mixture thereby increasing hair thickness to reduce hair breakage and damage to the colored hair.

17. A method of reducing hair frizziness in colored hair comprising the steps of:
applying the hair colorant mixture of claim 12 to a surface of the hair in need of treatment; and
rinsing the hair to remove any excess hair colorant mixture thereby causing the hair to exhibit less frizz.

18. A method for reducing breakage of or damage to hair, the method comprising the steps of:
applying to a surface of the hair in need of treatment a hair treatment composition, the hair treatment composition comprising:
one or more of a shampoo, conditioner, mask, or paste; and
a keratin protein filler composition.
wherein, upon application, hair thickness is increased to reduce hair breakage and damage to the hair.

19. The method of claim 18, further comprising the step of rinsing the hair to remove the hair treatment composition.

20. The method of claim 19, wherein the hair treatment composition is left in contact with the hair for up to ten minutes prior to rinsing.
